(19) United States
(12) Patent Application Publication
(10) Pub. No.: US 2007/0098816 A1
(43) Pub. Date: May 3, 2007

(54) Pharmaceutical composition containing histone deacetylase inhibitor

(76) Inventors: Osamu Nakanishi, Hyogo (JP); Tatsuo Sugawara, Berlin (DE); Hideyuki Migita, Richmond, CA (US); Yasuhiro Matsuba, Hyogo (JP)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/558,208

(22) PCT Filed: May 26, 2004
(86) PCT No.: PCT/JP04/07562

§ 371 Date: Dec. 4, 2006

(30) Foreign Application Priority Data
May 26, 2003 (JP) 2003-148073

(51) Int. Cl. A61K 33/24; A61K 31/7048; A61K 31/44; A61K 31/325

(52) U.S. Cl. 424/649; 514/357; 514/27; 514/485

(57) ABSTRACT

An anticancer drug having a synergistic effect by combined use of a histone acetylase derivative such as N-(2-aminophenyl)-4-[N-(pyridin-3-ylmethoxycarbonyl) amninomethyl]benzamide (MS-275) and another anticancer active substance.

### TECHNICAL FIELD

The present invention relates to a pharmaceutical composition or drug combination for treatment of cancer comprising a histone deacetylase inhibitor and another anticancer active substance.

### BACKGROUND ART

At the present time, cancer is the first leading cause of death. Up until now, many researches on cancer have been conducted and tremendous money and time have been spended on these researches. However, despite research in methods of treatment spanning diverse fields such as surgery, radiotherapy, and thermotherapy, cancer has not been overcome. Among these, chemotherapy is a major sector and many anticancer drugs have been researched. For example, as chemotherapy drugs for cancer, cisplatin, etoposide, 5-fluorouracil, gemcitabine, paclitaxel, docetaxel, carboplatin, oxaliplatin, doxorubicin, vinblastin, etc. have been. used.

Japanese Unexamined Patent Publication (Kokai) No. 10-152462 discloses a benzamide derivative. The following fact is disclosed; said benzamide derivative has a differentiation inducing action, is useful as a pharmaceutical for the treatment or alleviation of malignant tumors, autoimmune diseases, skin diseases, and parasitic infection, is particularly effective as an anticancer drug, and is effective against hematopoietic cancers and solid cancers.

Patent Document 1

Japanese Unexamined Patent Publication (Kokai) No. 10-152462

### DISCLOSURE OF THE INVENTION

However, anticancer drugs have limitation at a dosage of a single drug due to their strong toxicity to normal cells. Except for some cancers, treatment by administration of a single drug is not enough to achieve a sufficient efficacy.

The present invention was made to reduce the toxicity posing a problem in current chemotherapy and achieve a high treatment effect.

Accordingly, the present invention provides a pharmaceutical composition or combination as active ingredients comprising:

(a) at least one of the benzamide derivatives represented by formula (1):

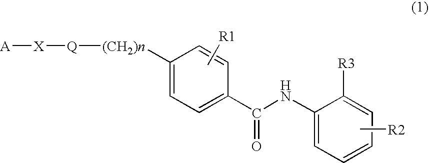

wherein A is an optionally substituted phenyl group or an optionally substituted heterocyclic group wherein the substituent(s) for the phenyl group or the heterocyclic group is (are) 1 to 4 substituents selected from the group consisting of a halogen atom, a hydroxyl group, an amino group, a nitro group, a cyano group, an alkyl group having 1 to 4 carbons, an alkoxy group having 1 to 4 carbons, an aminoalkyl group having 1 to 4 carbons, an alkylamino group having 1 to 4 carbons, an acyl group having 1 to 4 carbons, an acylamino group having 1 to 4 carbons, an alkylthio group having 1 to 4 carbons, a perfluoroalkyl group having 1 to 4 carbons, a perfluoroalkyloxy group having 1 to 4 carbons, a carboxyl group, an alkoxycarbonyl group having 1 to 4 carbons, a phenyl group and a heterocyclic group;

X is a bond or a moiety having a structure selected from those illustrated in formula (2):

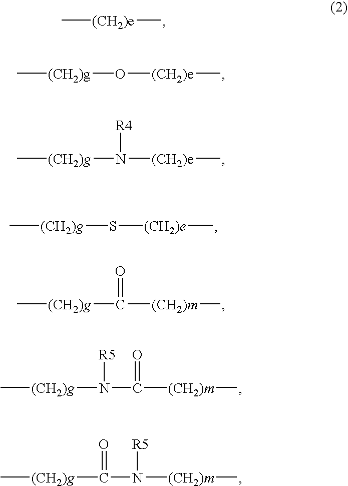

wherein e is an integer of 1 to 4; g and m are independently an integer of 0 to 4; R4 is a hydrogen atom or an optionally substituted alkyl group having 1 to 4 carbons, or the acyl group represented by formula (3)

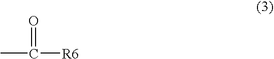

wherein R6 is an optionally substituted alkyl group having 1 to 4 carbons, a perfluoroalkyl group having 1 to 4 carbons, a phenyl group or a heterocyclic group; R5 is a hydrogen atom or an optionally substituted alkyl group having 1 to 4 carbons;

n is an integer of 0 to 4, provided that when X is a bond, n is not zero;

Q is a moiety having a structure selected from those illustrated in formula (4)

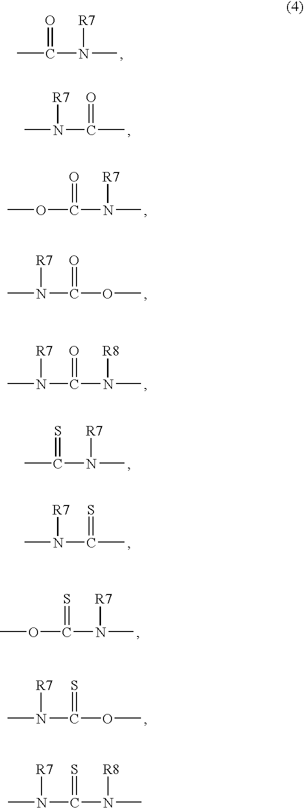

wherein R7 and R8 are independently a hydrogen atom or an optionally substituted alkyl group having 1 to 4 carbons;

R1 and R2 are independently a hydrogen atom, a halogen atom, a hydroxyl group, an amino group, an alkyl group having 1 to 4 carbons, an alkoxy group having 1 to 4 carbons, an aminoalkyl group having 1 to 4 carbons, an alkylamino group having 1 to 4 carbons, an acyl group having 1 to 4 carbons, an acylamino group having 1 to 4 carbons, an alkylthio group having 1 to 4 carbons, a perfluoroalkyl group having 1 to 4 carbons, a perfluoroalkyloxy group having 1 to 4 carbons, a carboxyl group or an alkoxycarbonyl group having 1 to 4 carbons;

R3 is a hydroxyl group or amino group or a pharmaceutically acceptable salt thereof as HDAC inhibiting substance, and

(b) at least one substance as another anti-cancer active substance selected from a group consisting of cisplatin, etoposide, camptothecin, 5-fluorouracil, gemcitabine, paclitaxel, docetaxel, carboplatin, oxaliplatin, doxorubicin and vinblastin.

The present invention further provides a cancer treatment kit comprising a pharmaceutical combination, which comprises:

(i) at least one of said ingredients (a) which is a histone deacetylase inhibiting substance,

(ii) at least one of said ingredients (b) which is another anti-cancer active substance, and

(iii) an instruction for administration schedule for simultaneous or sequential administration according to a kind of cancer (for sequential administration to a patient at periodic intervals).

The “pharmaceutical combination” in the present invention means a combination of an ingredient (a) which is a histone deacetylase inhibiting substance and an ingredient (b) which is another anti-cancer active substance, wherein the ingredient (a) and the ingredient (b) are administered simultaneously or at different times (or sequentially).

The present invention includes a method of treatment of cancer comprising administering said ingredient (a) and said ingredient (b) to patients simultaneously or at different times (or sequentially). In this situation, an administration sequence of said ingredient (a) and said ingredient (b) is appropriately selected according to a kind of cancer and kinds of said ingredient (a) and said ingredient (b). Further, the present invention also includes use of said ingredient (a) and said ingredient (b) for producing a pharmaceutical composition or drug combination of the present invention for treating cancer and use of said ingredient (a) and said ingredient (b) for producing the kit of the present invention.

The benzamide derivative which is a histone deacetylase inhibiting substance or pharmaceutically acceptable salts thereof is preferably selected from represented by the following formulas (5) to (8):

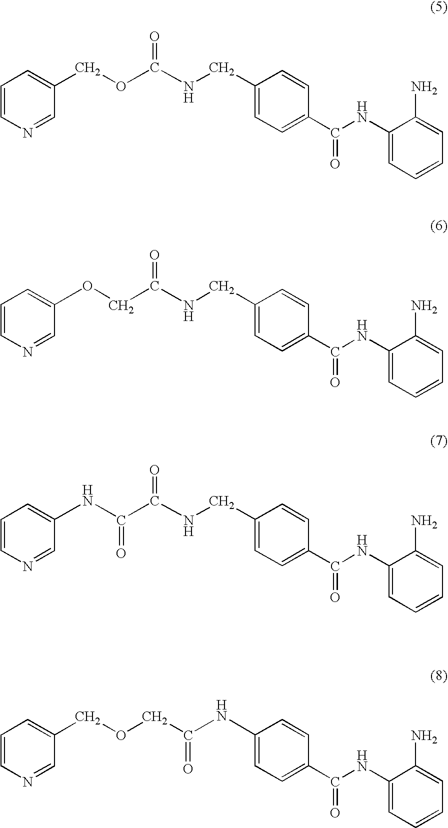

More preferably, the benzamide derivative is represented by the following formula (5) or pharmaceutically acceptable salt thereof:

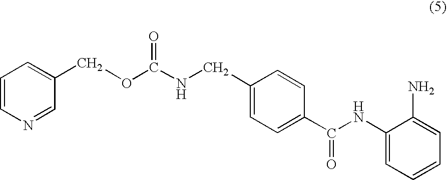

In the pharmaceutical combination or composition in the present invention, said ingredient (b) which is another anti-cancer active substance is preferably cisplatin, more preferably the combination or composition which is for treatment of colon cancer, non-small cell lung cancer, ovarian cancer or pancreatic cancer.

Further, in the pharmaceutical combination or composition in the present invention, said ingredient (b) which is another anti-cancer active substance is preferably etoposide, more preferably the combination or composition which is for treatment of ovarian cancer.

Further, in the pharmaceutical combination or composition in the present invention, said ingredient (b) which is another anti-cancer active substance is preferably camptothecin, more preferably the combination or composition which is for treatment of colon cancer, non-small cell lung cancer, ovarian cancer or pancreatic cancer.

Further, in the pharmaceutical combination or composition in the present invention, said ingredient (b) which is another anti-cancer active substance is preferably 5-fluorouracil, more preferably the combination or composition which is for treatment of breast cancer or colon cancer.

Further, in the pharmaceutical combination or composition in the present invention, said ingredient (b) which is another anti-cancer active substance is preferably gemcitabine, more preferably the combination or composition which is for treatment of non-small cell lung cancer, colon cancer or ovarian cancer.

Further, in the pharmaceutical combination or composition in the present invention, said ingredient (b) which is another anti-cancer active substance is preferably paclitaxel, more preferably the combination or composition which is for treatment of breast cancer, prostate cancer or ovarian cancer.

Further, in the pharmaceutical combination or composition in the present invention, said ingredient (b) which is another anti-cancer active substance is preferably docetaxel, more preferably the combination or composition which is for treatment of non-small cell lung cancer, ovarian cancer, pancreatic cancer or prostate cancer.

Further, in the pharmaceutical combination or composition in the present invention, said ingredient (b) which is another anti-cancer active substance is preferably carboplatin, more preferably the combination or composition which is for treatment of non-small cell lung cancer, ovarian cancer or pancreatic cancer.

Further, in the pharmaceutical combination or composition in the present invention, said ingredient (b) which is another anti-cancer active substance is preferably oxaliplatin, more preferably the combination or composition which is for treatment of colon cancer or ovarian cancer.

Further, in the pharmaceutical combination or composition in the present invention, said ingredient (b) which is another anti-cancer active substance is preferably doxorubicin, more preferably the combination or composition which is for treatment of ovarian cancer.

Further, in the pharmaceutical combination or composition in the present invention, said ingredient (b) which is another anti-cancer active substance is preferably vinbiastin, more preferably the combination or composition which is for treatment of non-small cell lung cancer.

Further, the pharmaceutical combination in the present invention is preferable, of which said ingredient (a) which is histone deacetylase inhibiting substance and said ingredient (b) which is another anti-cancer active substance are sequentially administered to patients.

Of the pharmaceutical combination, said ingredient (b) which is another anti-cancer active substance is preferably paclitaxel. As the administration sequence thereof, it is preferable to administer paclitaxel and then said ingredient (a) which is a histone deacetylase inhibiting substance. The pharmaceutical combination for treatment of breast cancer or ovarian cancer is more preferable.

Further, of the pharmaceutical combination, said ingredient (b) which is another anti-cancer active substance is preferably cisplatin. As the administration sequence thereof, it is preferable to administer said ingredient (a) which is a histone deacetylase inhibiting substance, and then cisplatin. The pharmaceutical combination for treatment of non-small cell lung cancer is more preferable. Or, the administration sequence thereof is preferably cisplatin, and then said ingredient (a) which is a histone deacetylase inhibiting substance. The pharmaceutical combination for treatment of colon cancer, non-small cell lung cancer, ovarian cancer or pancreatic cancer is more preferable.

Further, of the pharmaceutical combination, said ingredient (b) which is another anti-cancer active substance is preferably gemcitabine. As the administration sequence thereof, it is preferable to administer said ingredient (a) which is a histone deacetylase inhibiting substance, and then gemcitabine. The pharmaceutical combination for treatment of non-small cell lung cancer is more preferable. Or, the administration sequence thereof is preferably gemcitabine, and then said ingredient (a) which is a histone deacetylase inhibiting substance. The pharmaceutical combination for treatment of colon cancer, non-small cell lung cancer, ovarian cancer or pancreatic cancer is more preferable.

Further, of the pharmaceutical combination, said ingredient (b) which is another anti-cancer active substance is preferably docetaxel. As the administration sequence thereof, it is preferable to administer docetaxel, and then said ingredient (a) which is a histone deacetylase inhibiting substance. The pharmaceutical combination for treatment of non-small cell lung cancer, ovarian cancer, pancreatic cancer or prostate cancer is more preferable.

Further, of the pharmaceutical combination, said ingredient (b) which is another anti-cancer active substance is preferably carboplatin. AS the administration sequence thereof, it is preferable to administer carboplatin, and then said ingredient (a) which is a histone deacetylase inhibiting substance. The pharmaceutical combination for treatment of non-small cell lung cancer, ovarian cancer, pancreatic cancer or prostate cancer is more preferable.

Further, of the pharmaceutical combination, said ingredient (b) which is another anti-cancer active substance is preferably oxaliplatin. As the administration sequence thereof, it is preferable to administer oxaliplatin, and then said ingredient (a) which is a histone deacetylase inhibiting substance. The pharmaceutical combination for treatment of colon cancer or ovarian cancer is more preferable.

Further, of the pharmaceutical combination, said ingredient (b) which is another anti-cancer active substance is preferably doxorubicin. As the administration sequence thereof, it is preferable to administer doxorubicin, and then said ingredient (a) which is a histone deacetylase inhibiting substance. The pharmaceutical combination for treatment of ovarian cancer is more preferable.

Further, of the pharmaceutical combination, said ingredient (b) which is another anti-cancer active substance is preferably vinblastin. As the administration sequence thereof, it is preferable to administer vinblastin, and then said ingredient (a) which is a histone deacetylase inhibiting substance. The pharmaceutical combination for treatment of non-small cell lung cancer is more preferable.

Further, of the pharmaceutical combination, said ingredient (b) which is another anti-cancer active substance is preferably 5-fluorouracil. As the administration sequence thereof, it is preferable to administer 5-fluorouracil, and then said ingredient (a) which is a histone deacetylase inhibiting substance. The pharmaceutical combination for treatment of colon cancer is more preferable.

In the pharmaceutical composition of the present invention, said ingredient (a) and said ingredient (b) may be made into the pharmaceutical composition using compound per se which are these active ingredients, may be made into the pharmaceutical composition using a preparation containing said ingredient (a) as an active ingredient and a preparation containing said ingredient (b) as an active ingredient, or may be made into the pharmaceutical composition using the compound per se which is either of said ingredient (a) or said ingredient (b) and a preparation of the other prepared in advance. And, in the pharmaceutical combination of the present invention, usually separately prepared preparations, that is, a preparation containing said ingredient (a) as an active ingredient and a preparation containing said ingredient (b) as an active ingredient, are administered simultaneously or at a different time (or consecutively).

### BRIEF DESCRIPTION OF DRAWINGS

FIG. 1 is a graph showing the principle of judgment of the existence of a synergistic action.

### BEST MODE FOR CARRYING OUT THE INVENTION

The present invention relates to a pharmaceutical composition or combination comprising a benzamide derivative represented by formula (1) which is a histone deacetylase inhibiting substance and another anticancer active substance.

As used herein, “1 to 4 carbons” means a carbon number per a single substituent; for example, for dialkyl substitution it means 2 to 8 carbons.

A heterocycle in the compound represented by formula (1) is a monocyclic heterocycle having 5 or 6 members containing 1 to 4 nitrogen, oxygen or sulfur atoms or a bicyclic-fused heterocycle. The monocyclic heterocycle includes pyridine, pyrazine, pyrimidine, pyridazine, thiophene, furan, pyrrole, pyrazole, isoxazole, isothiazole, imidazole, oxazole, thiazole, piperidine, piperazine, pyrrolidine, quinuclidine, tetrahydrofuran, morpholine, thiomorpholine and the like. The bicyclic fused heterocycle includes quinoline; isoquinoline; naphthyridine; fused pyridines such as furopyridine, thienopyridine, pyrrolopyridine, oxazolopyridine, imidazolopyridine and thiazolopyridine; benzofuran; benzothiophene; benzimidazole and the like. A halogen may be fluorine, chlorine, bromine or iodine. An alkyl having 1 to 4 carbons includes methyl, ethyl, n-propyl, isopropyl, n-butyl, isobutyl, sec-butyl and tert-butyl.

An alkoxy having 1 to 4 carbons includes methoxy, ethoxy, n-propoxy, isopropoxy, allyloxy, n-butoxy, isobutoxy, sec-butoxy, tert-butoxy and the like.

An aminoalkyl having 1 to 4 carbons includes aminomethyl, 1-aminoethyl, 2-aminopropyl and the like. An alkylamino having 1 to 4 carbons includes N-methylamino, N,N-dimethylamino, N,N-diethylamino, N-methyl-N-ethylamino, N,N-diisopropylamino and the like. An acyl having 1 to 4 carbons includes acetyl, propanoyl, butanoyl and like. An acylamino having 1 to 4 carbons includes acetylamino, propanoylamino, butanoylamino and the like. An alkylthio having 1 to 4 carbons includes methylthio, ethylthio, propylthio and the like. A perfluoroalkyl having 1 to 4 carbons includes trifluoromethyl, pentafluoroethyl and the like. A perfluoroalkyloxy having 1 to 4 carbons includes trifluoromethoxy, pentafluoroethoxy and the like. An alkoxycarbonyl having 1 to 4 carbons includes methoxycarbonyl and ethoxycarbonyl. An optionally substituted alkyl having 1 to 4 carbons includes methyl, ethyl, n-propyl, isopropyl, n-butyl, isobutyl, sec-butyl and tert-butyl and these having 1 to 4 substituents selected from the group consisting of a halogen, hydroxyl, amino, nitro, cyano, phenyl and a heterocycle.

A pharmaceutically acceptable salt of ingredient (a) as histone deacetylase inhibiting substance of this invention includes salts with an inorganic acid such as hydrochloric acid, hydrobromic acid, sulfuric acid and phosphoric acid; and with an organic acid such as acetic acid, lactic acid, tartaric acid, malic acid, succinic acid, fumaric acid, maleic acid, citric acid, benzoic acid, trifluoroacetic acid, p-toluenesulfonic acid and methanesulfonic acid.

The ingredient (a) which is a histone deacetylase inhibiting substance of this invention may be produced in accordance with the process of Japanese unexamined patent publication (Kokai) No. 10-152462. And, the ingredient (b) which is another anti-cancer active substance is commercially available or can be produced by known methods.

The pharmaceutical composition or combination of this invention is useful for cancer treatment. The composition itself may be used in the form of a general pharmaceutical formulation. And of the combination the ingredients (a) and (b) may be used in the form of a general pharmaceutical formulation.

The pharmaceutical composition comprising the active ingredient (a) and (b) is prepared with a generally used diluent or excipient such as filler, extender, binder, moisturizing agent, disintegrator, surfactant and lubricant. And the pharmaceutical combination is prepared by independent active ingredients, with a generally used diluent or excipient such as filler, extender, binder, moisturizing agent, disintegrator, surfactant and lubricant. The pharmaceutical formulation may have a variety of dosage forms such as tablet, pill, powder, solution, suspension, emulsion, granule, capsule, injection (e.g., solution, suspension) and suppository.

For preparing tablets, a variety of carriers well-known in the art may be used. Such a carrier includes excipients such as lactose, glucose, starch, calcium carbonate, kaoline, crystalline cellulose and silicic acid; binders such as water, ethanol, propanol, simple syrup, glucose solution, starch solution, gelatin solution, carboxymethyl cellulose, shellac, methyl cellulose and polyvinyl pyrrolidone; disintegrators such as dried starch, sodium alginate, powdered agar, calcium carmelose, starch and lactose; disintegration retarders such as sucrose, cocoa butter and hydrogenated oil; absorption promoters such as quaternary ammonium base and sodium lauryl sulfate; moisturizing agents such as glycerin and starch; adsorbents such as starch, lactose, kaoline, bentonite, colloidal silicic acid; and glidants such as talc, stearates and polyethylene glycol. The tablet-may be, if necessary, one coated with a common coating; for example, sugar-coated tablet, gelatin-coated tablet, enteric coated tablet, film-coated tablet, double-layer tablet and multilayer tablet.

In forming pills, a variety of carriers well-known in the art may be used. Such a carrier includes excipients such as crystalline cellulose, lactose, starch, hydrogenated vegetable oil, kaoline and talc; binders such as powdered acacia, powdered tragacanth gum and gelatin; disintegrators such as calcium carmelose and agar.

Capsule may be prepared by blending an active ingredient with a variety of the above carriers as usual and filling the resulting blend into, for example, a hard or soft gelatin capsule or the like.

For preparing injection, solution, emulsion and suspension are sterilized and preferably isotonic with blood. It may be prepared using diluents commonly used in the art; for example, water, ethanol, macrogol, propylene glycol, ethoxylated isostearyl alcohol, polyoxyisostearyl alcohol and polyoxyethylene sorbitan fatty acid esters. The pharmaceutical preparation may contain sodium chloride necessary to prepare an isotonic solution, glucose or glycerin, as well as usual solubilizers, buffers and soothing agents.

Suppository may be formed using a variety of well-known carriers; for example, semi-synthetic glyceride, cocoa butter, higher alcohols, higher alcohol esters and polyethylene glycol.

Furthermore, the pharmaceutical formulation may contain coloring agents, preservatives, perfumes, flavors, sweeteners and/or other drugs.

The volume ratio of the active ingredients (b) to (a) to be included in the pharmaceutical composition of the present invention is not limited and is appropriately selected from a broad range of the volume ratios. In the case of cisplatin, the molar ratio is 0.001 to 10000, preferably 0.01 to 1000, to 1 of the benzamide derivative (said ingredient (a)). In the case of etoposide, the molar ratio is 0.001 to 10000, preferably 0.01 to 1000, to 1 of the benzamide derivative.

In the case of camptothecin, the molar ratio is 0.00001 to 10, preferably 0.0001 to 1, to 1 of the benzamide derivative (said ingredient (a)). In the case of 5-fluorouracil, the molar ratio is 0.01 to 100000, preferably 0.1 to 10000, to 1 of the benzamide derivative. In the case of gemcitabine, the molar ratio is 0.00001 to 100, preferably 0.0001 to 10, to 1 of the benzamide derivative (said ingredient (a)). In the case of paclitaxel, the molar ratio is 0.000001 to 0.01, preferably 0.00001 to 0.001, to 1 of the benzamide derivative (said ingredient (a)).

In the case of docetaxel, the molar ratio is 0.0000001 to 1, preferably 0.000001 to 0.1, to 1 of the benzamide derivative (said ingredient (a)).

In the case of carboplatin, the molar ratio is 0.001 to 10000, preferably 0.01 to 1000, to 1 of the benzamide derivative (said ingredient (a)).

In the case of oxaliplatin, the molar ratio is 0.001 to 10000, preferably 0.01 to 1000, to 1 of the benzamide derivative (said ingredient (a)).

In the case of doxorubicin, the molar ratio is 0.000001 to 1, preferably 0.00001 to 0.1, to 1 of the benzamide derivative (said ingredient (a)).

In the case of vinblastin, the molar ratio is 0.000001 to 1, preferably 0.00001 to 0.1, to 1 of the benzamide derivative (said ingredient (a)).

An administration route of the pharmaceutical composition or combination is not limited, and selected depending on their dosage form, patient's age, sex, severity of disease and other conditions. For example, tablet, pill, solution, suspension, emulsion, granule and capsule may be orally administered; injection may be intravenously administered solely or in combination with a common infusion fluid such as glucose, amino acids and the like, or if necessary, intramuscularly, subcutaneously or intraperitoneally as a sole preparation. Suppository may be intrarectally administered.

Dose of the pharmaceutical composition or combination of this invention may be selected, depending on their dosage form, patient's age, sex and severity of disease, and other conditions, as appropriate, and the amount of the active ingredients in the composition may be generally about 0.0001 to 1000 mg/kg a day. It is preferable that a unit dosage form may contain about 0.001 to 1000 mg of the active ingredient(s).

Further, in the case of pharmaceutical combinations, the amount of the active ingredient of the benzamide derivative (said ingredient (a)) may be about 0.0001 to 1000 mg per kg body weight. In the case of cisplatin, the amount may be about 0.01 to 50 mg per kg body weight. In the case of etoposide, the amount may be about 0.1 to 10 mg per kg body weight. In the case of camptothecin, the amount may be about 0.1 to 10 mg per kg body weight.

In the case of 5-fluorouracil, the amount may be about 0.1 to 200 mg per kg body weight.

In the case of gemcitabine, the amount may be about 1 to 300 mg per kg body weight. In the case of paclitaxel, the amount may be about 0.1 to 100 mg per kg body weight.

In the case of docetaxel, the amount may be about 0.1 to 50 mg per kg body weight.

In the case of carboplatin, the amount may be about 0.2 to 100 mg per kg body weight.

In the case of oxaliplatin, the amount may be about 0.1 to 50 mg per kg body weight.

In the case of doxorubicin, the amount may be about 0.1 to 50 mg per kg body weight.

In the case of vinblastin, the amount may be about 0.01 to 5 mg per kg body weight.

For administration of pharmaceutical combinations, in the case of simultaneous administration, the first active ingredient and the second active ingredient are administered without any time interval. In the case of administration at different times (consecutively), it is preferable to administer the first active ingredient and then administer the second active ingredient half a day to 60 days later.

### EXAMPLES

Next, the present invention will be explained with examples more specifically.

### Examples. Confirmation of Synergistic Effect Between Histone Deacetylase Inhibitor and Known Anticancer Active Substances on Cancer Cell Proliferation

The synergistic effects in combined use of the histone deacetylase inhibitor of the present invention and various types of known anticancer active substances on various types of cancer cell lines were confirmed by the examples.

Test Substances

As the histone deacetylase inhibitor of the present invention, N-(2-aminophenyl)4-[N-(pyridin-3-ylmethoxycarbonyl)aminomethyl]benzamide (MS-275) represented by the following formula (5) was used.

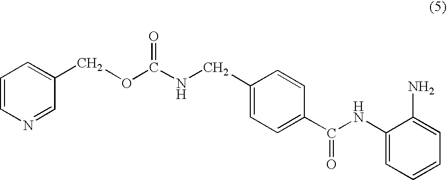

And, as known anticancer activity substances used in conjunction with the above MS-275 compound, paclitaxel (PTX), camptothecin (CPT), etoposide (VP-16), cisplatin (CDDP), gemcitabine (GEM), 5-fluorouracil (5-FU), docetaxel (DTX), carboplatin (CBDCA), oxaliplatin (OXP), doxorubicin (DOX), or vinblastin (VBL) was used.

Tested Cancer Cells

As the tested cancer cells, the following cell lines were used:

Colon cancer cell line: HT-29 and/or HCT116;

Non-small cell lung cancer cell line: NCI-H522, A549, Calu-1, Calu-3, NCI-H23, and/or NCI-H460;

Ovarian cancer cell line: SK-OV-3 and/or OVCAR-3;

Pancreatic cancer cell line: PANC-1 and/or Capan-1;

Breast cancer cell line: PC-3 and/or LNcaP.

Methods of Combined Use

In experiments, to evaluate the combined effect of the MS-275 which is a histone deacetylase inhibitor and another known anticancer active substance, (i) effects of the MS-275 alone, (ii) effects of another known anticancer active substance, and (iii) effects from combined use of the MS-275 and another anticancer active substance were measured. For the measurement of the effects of (iii), the following two types of methods were used.

Simultaneously Combined Use:

In this method, the test cancer cells were incubated for 72 to 120 hours in a medium containing a mixture of MS-275 and another known anticancer active substance, and then the surviving cancer cells were measured.

Consecutively Combined Use:

In this method, the test cancer cells were incubated for 24 hours in a medium containing one of the test substances, and the medium containing said test substance was aspirated at this point of time. Then the cells were incubated for 24 hours in a medium containing the other of the test substances, the medium containing said test substance was aspirated at this point of time, then the cells were incubated for another 72 hours in a medium not containing the test substances, and then the surviving cancer cells were measured. In the consecutively combined use, the MS-275 was made to act in the first 24 hours and the other known anticancer active substance was made to act in the succeeding 24 hours. And in the reversed order of what was made to act this experiment was performed. Further, in the single administration control for the combined use, the test substance was made to act in only the initial 24 hours or the succeeding 24 hours. In another 24 hour period and the final 72 hours, the cells were incubated in the absence of the test substance, and then the surviving cancer cells were measured.

Method of Measurement of Surviving Cancer Cells

After the above treatment (incubation) of the cancer cells by the test substances was ended, the surviving cells were measured by one of the following two methods.

Neutral Red Assay:

In this measurement method the following property is utilized; only surviving cells can take a water soluble dye, Neutral Red, into the cells. The above treatment of cancer cells by the test substance was performed in wells. A Neutral Red solution (1 mg/ml in PBS) was added into the wells after the end of the treatment (incubation). The incubation at 37° C. for one hour allowed the Neutral Red to be taken into the cells. The solution was aspirated and 100% ethanol and 0.1M NaH₂PO₄ were added to the wells. The Neutral Red taken into the cells was extracted from the cells and then the extracted Neutral Red was measured by a microplate reader at 540 nm.

MTS Assay:

This method is to investigate cell survivability by utilizing the fact that MTS (3-(4,5-dimethylthiazol-2-yl)-5-(3-carboxymethoxyphenol)-2-(4-sulfonyl)-2H-tetrazoliumm) is metabolized to formazan by mitochondria dehydrogenase existing in surviving cells. In this method the experiment was performed using a Cell Titer 96 (trademark) aqueous one solution cell proliferation assay of Promega in accordance with the instructions attached to the reagents.

Combined Ratio of Test Substances and Judament of Synergism

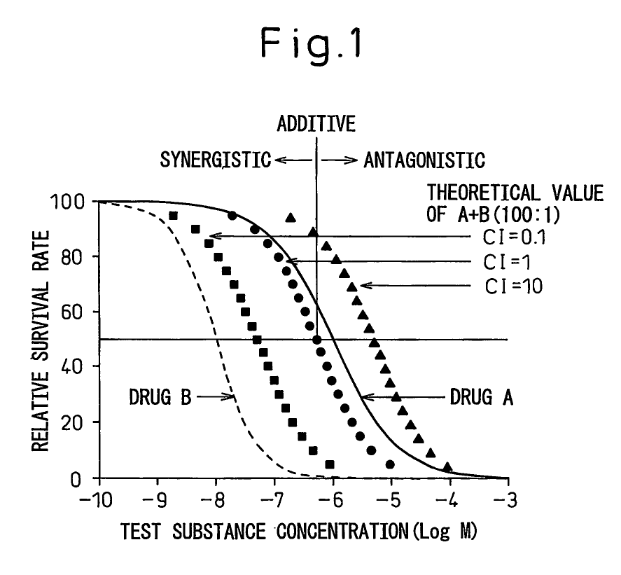

The combined ratio of the test substances was determined as follows: In the graph of FIG. 1, the abscissa shows the log (Log M) of the concentrations of the test substances, and the ordinate shows the relative survival rate in the case indexed to the surviving tested cancer cells in the case of zero concentration of test substances. Graphs of the concentration of the test substances and the relative survival rate of the tested cancer cells in the case of the test substances alone were made. The concentrations of the test substances in the case of relative survival rates of 50%, IC₅₀, were calculated.

Regarding the IC₅₀'s of the test substances A and B for which the existence of a synergistic effect was desired to be learned, in the case that the IC₅₀ of the test substance A was 1 μM and 0.01 μM as the IC₅₀ of the test substance B was 0.01 μM, since the anticancer effect of the test substance B was 100 times that of the test substance A, the combined ratio of the test substance A and test substance B was made 100:1. This ratio was kept constant across the various total concentrations of the test substances. However, the IC₅₀ of a test substance differed according to the tested cancer cells, so the combined ratio needed to be determined for each test substance and for each type of tested cancer cells.

In FIG. 1, the “concentration-survival rate curve” of the test substance A was shown in a solid line, and the “concentration-survival rate curve” of the test substance B was shown in a dotted line. Further, given that the test substance A and test substance B were used in a constant ratio (for example, 100:1) and at various total concentrations and that the combined effect of the test substances was “additive”, a “concentration-survival rate curve” could be drawn for the case of combined use by calculation. For example, in FIG. 1, this could be shown in a series of black dots.

On the other hand, an actual “concentration-survival rate curve” could be drawn by calculating from the actually measured values in the case of use of the test substance A and test substance B at a constant ratio (for example, 100:1) but at various total concentrations. When the curve is present at the left side from the “concentration-survival rate curve” drawn by calculation under the assumption of “additive” as shown for example by a series of black squares in FIG. 1, the combined effects of the test substance A and the test substance B were judged to be “synergistic”. Meanwhile, when the actual “concentration-survival rate curve” was drawn at the right side from the “concentration-survival rate curve” drawn by calculation under the assumption of “additive” as shown for example by a series of black triangles in FIG. 1, the combined effects of the test substance A and the test substance B were judged to be “antagonistic”.

In actuality, the combination index (CI) was calculated from the measurement results by the method described in Chou TC et al., Adv. Enzyme Regul. 22: 27-55 (1984) (Quantitative analysis of dose-effect relationships: the combined effects of multiple drugs or enzyme inhibitors). In this case, when the combined effects of the test substance A and test substance B were additive, CI=1. When CI was less than 1, the effects were synergistic. When CI was more than one, the effects were antagonistic. Further, the following were judged; the smaller a value less than 1 was the higher the “synergism” was. And the greater a value more than 1 was, the higher the “antagonism” was.

Further, the relationship between the range of the CI value and the degree of synergism and antagonism is expressed as follows:

TABLE 1

| Range of CI value | Symbol | Description |
|---|---|---|
| <0.1 | +++++ | Very strongly synergistic |
| 0.1 to 0.3 | ++++ | Strongly synergistic |
| 0.3 to 0.7 | +++ | Synergistic |
| 0.7 to 0.85 | ++ | Moderately synergistic |
| 0.85 to 0.9 | + | Slightly synergistic |
| 0.9 to 1.1 | ± | Additive |
| 1.1< | − | Antagonistic |

Results

The ratios between MS-275 and other anticancer active substances with respect to each tested cancer cell line in the case of simultaneous combined use are as follows:

TABLE 2
Ratio of MS-275 and Other Anticancer Active
Substances (X) in Simultaneous Combined Use

| | | Time | Ratio (MS-275:X) | | | | | |
|---|---|---|---|---|---|---|---|---|
| Cancer cell line | | (hr) | PTX | CPT | VP-16 | CDDP | GEM | 5-FU |
| Colon | HT-29 | 72 | | 30:1 | | 1:5 | 5:1 | 1:10 |
| cancer | HCT116 | 72 | | 50:1 | 1:1 | 1:10 | 100:1 | 1:10 |
| Non-small | NCI-H522 | 72 | 200:1 | | | | 500:1 | |
| cell lung | | 120 | 400:1 | | | | 2000:1 | |
| cancer | A549 | 72 | 100:1 | | | 1:10 | 40:1 | |
| Ovarian | SK-OV-3 | 72 | 1000:1 | 100:1 | 1:1 | 1:2 | | |
| cancer | | 120 | 1000:1 | 100:1 | 1:1 | 1:2 | | |
| | OVCAR-3 | 120 | 1000:1 | 100:1 | 4:1 | 1:1 | 200:1 | |
| Pancreatic | PANC-1 | 72 | 2000:1 | 200:1 | | 1:1 | 200:1 | 1:1 |
| cancer | | 120 | 2000:1 | 400:1 | | 1:1 | 200:1 | 1:1 |
| Breast | MCF-7 | 72 | 400:1 | | | | | 1:10 |
| cancer | | 120 | 400:1 | | | | | 1:10 |
| Prostate | PC-3 | 72 | 100:1 | | 1:40 | | | |
| cancer | | 120 | 10:1 | | 1:50 | | | |

The results in the case of simultaneous combined use are as follows:

TABLE 3
Synergistic Effect in Combined Use of MS-275 and Other Anticancer
Active Substances in Simultaneous Combined Use

| | | Time | Other anticancer active substance | | | | | |
|---|---|---|---|---|---|---|---|---|
| Cancer cell line | | (hr) | PTX | CPT | VP-16 | CDDP | GEM | 5-FU |
| Colon | HT-29 | 72 | | − | | − | − | − |
| cancer | | 72 | | | | | | +++ |
| | HCT116 | 72 | | − | − | − | − | − |
| Non-small | NCI-H522 | 72 | − | | | | ± | |
| cell lung | | 120 | − | | | | − | |
| cancer | | 72 | | | | ± | | |
| | A549 | 72 | − | | | − | − | |
| | | 72 | | | | +++ | | |
| | Calu-1 | 72 | | | | +++ | | |
| | Calu-3 | 72 | | | | +++ | | |
| | A-427 | 72 | | | | − | | |
| | NCI-H23 | 72 | | | | +++ | | |
| | NCI-H358 | 72 | | | | ± | | |
| | NCI-H460 | 72 | | | | +++ | | |
| Ovarian | SK-OV-3 | 72 | − | − | +++ | ++ | | |
| cancer | | 120 | − | − | ± | − | | |
| | OVCAR-3 | 120 | − | − | − | − | − | |
| Pancreatic | PANC-1 | 72 | − | +++ | | ++ | +++ | − |
| cancer | | 120 | − | − | | ++ | − | − |
| Breast | MCF-7 | 72 | | | | | | +++ |
| cancer | | 120 | − | | | | | ++ |
| Prostate | PC-3 | 72 | − | | − | | | |
| cancer | | 120 | ++ | | − | | | |

As explained above, the combined effects of MS-275 and another known anticancer drug PTX, CPT, VP-16, GEM, or 5-FU were detected in specific cancer cells. Further, the combined effects of MS-275 and CDDP were detected in a broad range of cancer cells.

Further, the results in the case of consecutive combined use are shown in Table 4 (combined use of MS-275 and PTX), Table 5 (combined use of MS-275 and GEM), Table 6 (combined use of MS-275 and CDDP), Table 7 (combined use of MS-275 and CPT), Table 8 (combined use of MS-275 and DTX), Table 9 (combined use of MS-275 and CBDCA), Table 10 (combined use of MS-275 and OXP), Table 11 (combined use of MS-275 and DOX), Table 12 (combined use of MS-275 and VBL), and Table 13 (combined use of MS-275 and 5-FU). Note that in these tables, “Ratio 275:XS” means the ratio of MS-275 and another anticancer active substance (X), while “275->X->f” indicates treatment by MS-275 in the initial treatment period of 24 hours, treatment by another anticancer active substance in the following treatment period of 24 hours, then incubation in a medium not containing the test substance for 72 hours. Further, “X->275->f” indicates treatment by another anticancer active substance in the initial treatment period of 24 hours, treatment by MS-275 in the following treatment period of 24 hours, then incubation in a medium not containing the test substance for 72 hours. Further, the numerical values showing the synergistic effect show the CI values.

TABLE 4
Synergistic Effect in Consecutive
Combined Use of MS-275 and PTX

| | | | | Order of consecutive | |
|---|---|---|---|---|---|
| | | | | combined use | |
| | | | Ratio | 275−> | X−> |
| Cancer cell line | | Time (hr) | 275:X | X−>f | 275−>f |
| Ovarian | SK-OV-3 | 24 + 24 + 72 | 1000:1 | 1.1< − | 0.76 ++ |
| cancer | | | | | |
| Breast | T-47D | 24 + 24 + 72 | 1000:1 | | 0.71 ++ |
| cancer | | | | | |

TABLE 5
Synergistic Effect in Consecutive
Combined Use of MS-275 and GEM

| | | | | Order of consecutive | |
|---|---|---|---|---|---|
| | | | | combined use | |
| | | | Ratio | 275−> | X−> |
| Cancer cell line | | Time (hr) | 275:X | X−>f | 275−>f |
| Colon | HT-29 | 24 + 24 + 72 | 200:1 | 1.1< − | 0.48 +++ |
| cancer | | | | | |
| Non-small | NCI-H522 | 24 + 24 + 72 | 200:1 | 0.75 ++ | 1.1< − |
| cell lung | NCI-H522 | 24 + 24 + 72 | 3000:1 | | 0.77 ++ |
| cancer | A549 | 24 + 24 + 72 | 100:1 | 1.1< − | 0.69 +++ |
| Ovarian | OVCAR-3 | 24 + 24 + 72 | 400:1 | 1.1 − | 0.54 +++ |
| cancer | SK-OV3 | 24 + 24 + 72 | 5000:1 | | 0.56 +++ |
| Pancreatic | PANC-1 | 24 + 24 + 72 | 50000:1 | | 0.59 +++ |
| cancer | | | | | |

TABLE 6
Synergistic Effect in Consecutive
Combined Use of MS-275 and CDDP

| | | | | Order of consecutive | |
|---|---|---|---|---|---|
| | | | | combined use | |
| | | | Ratio | 275−> | X−> |
| Cancer cell line | | Time (hr) | 275:X | X−>f | 275−>f |
| Colon | HCT116 | 24 + 24 + 72 | 1:8 | 0.63 +++ | 0.95 ± |
| cancer | HT-29 | 24 + 24 + 72 | 4:1 | | 0.89 + |
| Non-small | NCI-H522 | 24 + 24 + 72 | 1:1 | 0.55 +++ | 0.69 +++ |
| cell lung | A549 | 24 + 24 + 72 | 1:4 | 0.66 +++ | 0.42 +++ |
| cancer | | | | | |
| Ovarian | SK-OV3 | 24 + 24 + 72 | 1:1 | 0.43 +++ | 0.57 +++ |
| cancer | OVCAR-3 | 24 + 24 + 72 | 1:1 | 0.77 ++ | 0.61 +++ |
| Pancreatic | PANC-1 | 24 + 24 + 72 | 8:1 | 0.96 ± | 0.45 +++ |
| cancer | Capan-1 | 24 + 24 + 72 | 1:1 | 0.53 +++ | 0.63 +++ |

TABLE 7
Synergistic Effect in Consecutive
Combined Use of MS-275 and CPT

| | | | | Order of consecutive | |
|---|---|---|---|---|---|
| | | | | combined use | |
| | | | Ratio | 275−> | X−> |
| Cancer cell line | | Time (hr) | 275:X | X−>f | 275−>f |
| Colon | HCT116 | 24 + 24 + 72 | 100:1 | 0.91 ± | 0.85 ++ |
| cancer | | | | | |
| Non-small | NCI-H522 | 24 + 24 + 72 | 100:1 | 0.31 +++ | 0.92 ± |
| cell lung | A549 | 24 + 24 + 72 | 25:1 | 1.1< − | 0.79 ++ |
| cancer | | | | | |
| Ovarian | OVCAR-3 | 24 + 24 + 72 | 200:1 | 1.05 ± | 0.26 ++++ |
| cancer | SK-OV3 | 24 + 24 + 72 | 2000:1 | | 0.72 ++ |
| Pancreatic | Capan-1 | 24 + 24 + 72 | 200:1 | 1.1< − | 0.49 +++ |
| cancer | | | | | |

TABLE 8
Synergistic Effect in Consecutive Combined
Use of MS-275 and DTX (Docetaxel)

| | | | | Order of consecutive | |
|---|---|---|---|---|---|
| | | | | combined use | |
| | | | Ratio | 275−> | X−> |
| Cancer cell line | | Time (hr) | 275:X | X−>f | 275−>f |
| Non-small | A549 | 24 + 24 + 72 | 10000:1 | | 0.87 + |
| cell lung | | | | | |
| cancer | | | | | |
| Ovarian | SK-OV3 | 24 + 24 + 72 | 20000:1 | | 0.87 + |
| cancer | | | | | |
| Pancreatic | Capan-1 | 24 + 24 + 72 | 3000:1 | | 0.87 + |
| cancer | | | | | |
| Prostate | PC-3 | 24 + 24 + 72 | 300:1 | | 0.89 + |
| cancer | | | | | |

TABLE 9
Synergistic Effect in Consecutive Combined Use
of MS-275 Compound and CBDCA (Carboplatin)

| | | | | Order of consecutive | |
|---|---|---|---|---|---|
| | | | | combined use | |
| | | | Ratio | 275−> | X−> |
| Cancer cell line | | Time (hr) | 275:X | X−>f | 275−>f |
| Non-small | A549 | 24 + 24 + 72 | 1:10 | | 0.31 +++ |
| cell lung | NCI-H522 | 24 + 24 + 72 | 1:2 | | 0.86 + |
| cancer | | | | | |
| Ovarian | SK-OV3 | 24 + 24 + 72 | 3:2 | | 0.59 +++ |
| cancer | | | | | |
| Pancreatic | Capan-1 | 24 + 24 + 72 | 1:1 | | 0.47 +++ |
| cancer | PANC-1 | 24 + 24 + 72 | 1:1 | | 0.30 ++++ |

TABLE 10
Synergistic Effect in Consecutive Combined
Use of MS-275 and OXP (Oxaliplatin)

| | | | | Order of consecutive | |
|---|---|---|---|---|---|
| | | | Ratio | combined use | |
| Cancer cell line | | Time (hr) | 275:X | 275−>X−>f | X−>275−>f |
| Colon | HT-29 | 24 + 24 + 72 | 5:1 | | 0.77 ++ |
| cancer | | | | | |
| Ovarian | SK-OV3 | 24 + 24 + 72 | 2:1 | | 0.83 ++ |
| cancer | | | | | |

TABLE 11
Synergistic Effect in Consecutive Combined
Use of MS-275 and DOX (Doxorubicin)

| | | | | Order of consecutive | |
|---|---|---|---|---|---|
| | | | Ratio | combined use | |
| Cancer cell line | | Time (hr) | 275:X | 275−>X−>f | X−>275−>f |
| Ovarian | SK-OV3 | 24 + 24 + 72 | 300:1 | | 0.86 + |
| cancer | | | | | |

TABLE 12
Synergistic Effect in Consecutive Combined
Use of MS-275 and VBL (Vinblastin)

| | | | | Order of consecutive | |
|---|---|---|---|---|---|
| | | | Ratio | combined use | |
| Cancer cell line | | Time (hr) | 275:X | 275−>X−>f | X−>275−>f |
| Non-small | A549 | 24 + 24 + 72 | 300:1 | | 0.89 + |
| cell lung | | | | | |
| cancer | | | | | |

TABLE 13
Synergistic Effect in Consecutive Combined
Use of MS-275 and 5-FU (5-Fluorouracil)

| | | | | Order of consecutive | |
|---|---|---|---|---|---|
| | | | Ratio | combined use | |
| Cancer cell line | | Time (hr) | 275:X | 275−>X−>f | X−>275−>f |
| Colon | HT-29 | 24 + 24 + 72 | 2:3 | | 0.79 ++ |
| cancer | | | | | |

In each case of each of the tested anticancer active substances, synergistic effects due to combined use with MS-275 were detected.

### INDUSTRIAL APPLICABILITY

As explained above, synergistic effects are recognized in in vitro tests between histone deacetylase inhibitors as represented by MS-275 and other various types of known anticancer active substances, so it is suggested that synergistic effects will be obtained in treatment for human cancer patient as well.

1. A pharmaceutical composition or a combination comprising, as active ingredients:
(a) at least one of the benzamide derivatives which is a histone deacetylase inhibiting substance, or a pharmaceutically acceptable salt thereof, represented by the following formula (1): 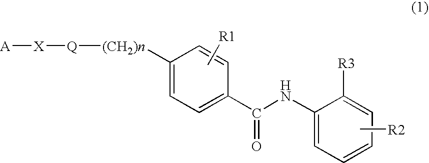
wherein A is an optionally substituted phenyl group or an optionally substituted heterocyclic group wherein the substituent(s) for the phenyl group or the heterocyclic group is (are) 1 to 4 substituents selected from the group consisting of a halogen atom, a hydroxyl group, an amino group, a nitro group, a cyano group, an alkyl group having 1 to 4 carbons, an alkoxy group having 1 to 4 carbons, an aminoalkyl group having 1 to 4 carbons, an alkylamino group having 1 to 4 carbons, an acyl group having 1 to 4 carbons, an acylamino group having 1 to 4 carbons, an alkylthio group having 1 to 4 carbons, a perfluoroalkyl group having 1 to 4 carbons, a perfluoroalkyloxy group having 1 to 4 carbons, a carboxyl group, an alkoxycarbonyl group having 1 to 4 carbons, a phenyl group and a heterocyclic group;
X is a bond or a moiety having a structure selected from those illustrated in formula (2): 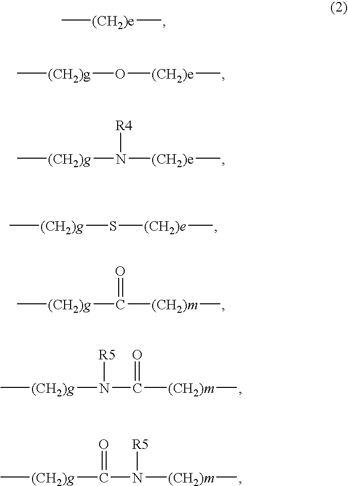
wherein e is an integer of 1 to 4; g and m are independently an integer of 0 to 4; R4 is a hydrogen atom or an optionally substituted alkyl group having 1 to 4 carbons, or the acyl group represented by formula (3) 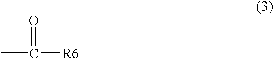
wherein R6 is an optionally substituted alkyl group having 1 to 4 carbons, a perfluoroalkyl group having 1 to 4 carbons, a phenyl group or a heterocyclic group; R5 is a hydrogen atom or an optionally substituted alkyl group having 1 to 4 carbons;
n is an integer of 0 to 4, provided that when X is a bond, n is not zero;
Q is a moiety having a structure selected from those illustrated in formula (4) 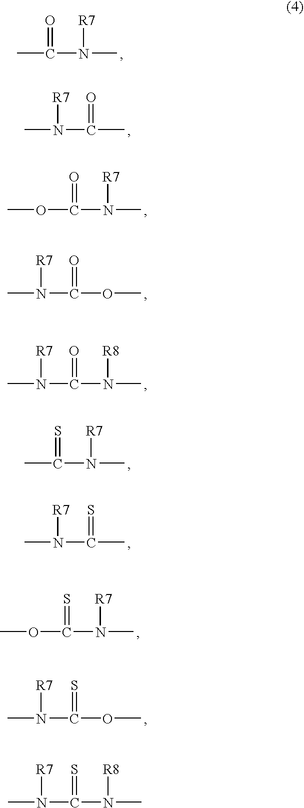
wherein R7 and R8 are independently a hydrogen atom or an optionally substituted alkyl group having 1 to 4 carbons;
R1 and R2 are independently a hydrogen atom, a halogen atom, a hydroxyl group, an amino group, an alkyl group having 1 to 4 carbons, an alkoxy group having 1 to 4 carbons, an aminoalkyl group having 1 to 4 carbons, an alkylamino group having 1 to 4 carbons, an acyl group having 1 to 4 carbons, an acylamino group having 1 to 4 carbons, an alkylthio group having 1 to 4 carbons, a perfluoroalkyl group having 1 to 4 carbons, a perfluoroalkyloxy group having 1 to 4 carbons, a carboxyl group or an alkoxycarbonyl group having 1 to 4 carbons;
R3 is a hydroxyl group or amino group, and
(b) at least one of the substances which is another anti-cancer active substance selected from a group consisting of cisplatin, etoposide, camptothecin, 5-fluorouracil, gemcitabine, paclitaxel, docetaxel, carboplatin, oxaliplatin, doxorubicin and vinblastin.

2. A pharmaceutical composition or a combination according to claim 1 wherein said benzamide derivative is selected from formulas (5) to (8) or a pharmaceutically acceptable salt thereof.

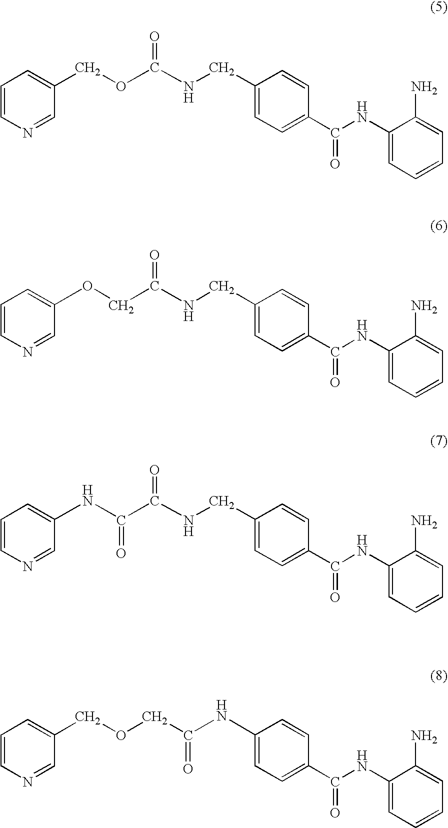

3. A pharmaceutical composition or a combination according to claim 1 wherein said benzamide derivative is represented by formula (5) or a pharmaceutically acceptable salt thereof.

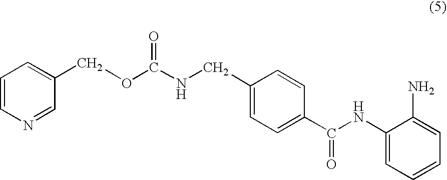

4. A pharmaceutical composition or a combination according to claim 1 wherein a substance selected from a group of substances consisting of said ingredient (b) which is another anti-cancer active substance is cisplatin.

5. A pharmaceutical composition or a combination according to claim 4, which is used for treatment of non-small cell lung cancer, ovarian cancer, colon cancer or pancreatic cancer.

6. A pharmaceutical composition or a combination according to claim 1 wherein a substance selected from a group of substances consisting of said ingredient (b) which is another anti-cancer active substance is etoposide.

7. A pharmaceutical composition or a combination according to claim 6, which is used for treatment of ovarian cancer.

8. A pharmaceutical composition or a combination according to claim 1 wherein a substance selected from a group of substances consisting of said ingredient (b) which is another anti-cancer active substance is camptothecin.

9. A pharmaceutical composition or a combination according to claim 8, which is used for treatment of non-small cell lung cancer, ovarian cancer, colon cancer or pancreatic cancer.

10. A pharmaceutical composition or a combination according to claim 1 wherein a substance selected from a group of substances consisting of said ingredient (b) which is another anti-cancer active substance is 5-fluorouracil.

11. A pharmaceutical composition or a combination according to claim 10, which is used for treatment of breast cancer or colon cancer.

12. A pharmaceutical composition or a combination according to claim 1 wherein a substance selected from a group of substances consisting of said ingredient (b) which is another anti-cancer active substance is gemcitabine.

13. A pharmaceutical composition or a combination according to claim 12, which is used for treatment of non-small cell lung cancer, ovarian cancer, colon cancer or pancreatic cancer.

14. A pharmaceutical composition or a combination according to claim 1 wherein a substance selected from a group of substances consisting of said ingredient (b) which is another anti-cancer active substance is paclitaxel.

15. A pharmaceutical composition or a combination according to claim 14, which is used for treatment of breast cancer, ovarian cancer or prostate cancer.

16. A pharmaceutical composition or a combination according to claim 1 wherein a substance selected from a group of substances consisting of said ingredient (b) which is another anti-cancer active substance is docetaxel.

17. A pharmaceutical composition or a combination according to claim 16, which is used for treatment of non-small cell lung cancers, ovarian cancer, pancreatic cancer and prostate cancer.

18. A pharmaceutical composition or a combination according to claim 1 wherein a substance selected from a group of substances consisting of said ingredient (b) which is another anti-cancer active substance is carboplatin.

19. A pharmaceutical composition or a combination according to claim 18, which is used for treatment of non-small cell lung cancer, ovarian cancer, or pancreatic cancer.

20. A pharmaceutical composition or a combination according to claim 1 wherein a substance selected from a group of substances consisting of said ingredient (b) which is another anti-cancer active substance is oxaliplatin.

21-63. (canceled)
